(19) United States
(12) Patent Application Publication
(10) Pub. No.: US 2009/0082680 A1
(43) Pub. Date: Mar. 26, 2009

(54) PERSONALIZED CARDIAC SAFETY RISK

(76) Inventors: Jan Mikael Totterman, Rochester, NY (US); Jean-Philip Couderc, Rochester, NY (US); Alexandra Latypova, Rochester, NY (US); Alex Zapesochny, Rochester, NY (US)

Correspondence Address: JAECKLE FLEISCHMANN & MUGEL, LLP, 190 Linden Oaks, ROCHESTER, NY, 14625-2812, US

(21) Appl. No.: 11/860,642

(22) Filed: Sep. 25, 2007

(51) Int. Cl. A61B 5/02

(52) U.S. Cl. 600/508

(57) ABSTRACT

A cardiac safety risk model for use in drug safety testing and/or pharmacological agent development is disclosed. The cardiac safety risk model has at least one baseline function based on at least one baseline biomarker. The cardiac safety risk model also has at least one follow-up function based on at least one follow-up biomarker. The cardiac safety risk model further has at least one overall function based on the at least one baseline function and the at least one follow-up function. A method of generating a cardiac safety risk model and a method of drug safety testing are also disclosed.

### FIELD

The claimed invention relates to biomarkers, and more specifically to cardiac repolarization biomarkers which may be used to generate a personalized cardiac safety risk model for drug safety testing and pharmacological agent development.

### BACKGROUND

The electrocardiogram (ECG) is based on the electrical activity of the heart muscle cells. In the resting stage, the inside of the cardiac cells has a negative charge compared to the outside of the cells. The resulting voltage difference between the internal and the external spaces of the cell membrane is called transmembrane potential. The discharging of this voltage is known as depolarization and is associated with the start of the contraction of the heart muscle cell fibers. After contraction of the ventricles, the heart muscle cells redevelop substantially the same voltage over the cell membrane. This recovery phase is called the repolarization process of the heart ventricles. An ECG measured from the skin surface measures a total electrical component created by the depolarization and repolarization of the heart's muscular cells.

The repolarization of the heart is made possible in part by ion channels within the myocardial cells of the heart which allow an ion current to redistribute charge. It is highly important that the regulation of the ion currents during the ventricular repolarization process occurs without interference, since a delay in this process or any other abnormalities can lead to a substantially increased risk for sudden cardiac death.

Recently, several important drugs have been removed from the market after it was revealed that these drugs were causing repolarization abnormalities in certain patients. The undesirable effect of these repolarization abnormalities was not fully identified in the existing safety assessment studies, which were mainly interested in a time interval which was not necessarily indicative of a change in repolarization morphology. The U.S. Food and Drug Agency (FDA) currently recommends that all pharmaceutical companies test the safety of all new compounds for their potential QT prolonging effect. The QT interval of an ECG encompasses a portion of the repolarization interval. Unfortunately, there is no standard for the measurement of a QT interval, and various techniques used to measure QT interval are not sensitive enough to properly identify a drug associated with a very small yet potentially deadly prolongation of the QT interval. Furthermore, as previously mentioned, QT interval does not quantify changes in the morphology (which includes both amplitude and duration) of the repolarization interval. Consequently, scientists have focused efforts on identifying other electrocardiographic markers besides QT prolongation for the identification of repolarization abnormalities, especially for the purposes of drug safety testing and pharmaceutical development.

Scientists trying to develop new electrocardiographic biomarkers have taken advantage of the fact that many of the heart's repolarization ion channels have been genetically identified, cloned, and functionally tested. Direct testing techniques, such as the “patch clamp” technique allow the electrical current of an isolated ion channel to be measured under various conditions, including in the presence of pharmaceuticals. Unfortunately, only about one quarter of the hundreds of types of ion channels identified by the sequencing of the human genome are currently able to be cloned and tested. One benefit, however of using ion-channel genome testing is that specific ion channels have been identified from the existing body of knowledge as being large contributors to the overall heart function. Scientists have been able to genetically identify patients with and without genetic mutations which affect such a single ion channel. By monitoring electrocardiogram (ECG) waveforms for the two types of patients and noting any differences in heart repolarization under the effects of a given pharmaceutical, various ECG-based bio-markers are able to be invented following analysis of the ECG waveforms. While useful, these ECG-based bio markers are not a complete measure of drug interaction risk since they are effectively a proxy or measure of an individual channel interaction. With over 400 possible types of ion channels and the cells of the heart having 100 to 1000 ion channels per cell, it is easy to see that the isolated patch-clamp ion channel testing and the population-based ECG-waveform biomarker testing does not allow a complete determination of drug safety risk, since characterization of drug safety risk is more complex than identifying a single ion channel and its response or a single ECG biomarker.

Therefore, it would be useful to have a method and system which moves beyond the individual biomarker and which is adaptable over time as the body of knowledge related to heart repolarization increases for the characterization of a personalized cardiac safety risk.

### SUMMARY

A cardiac safety risk model for use in drug safety testing and/or pharmacological agent development is disclosed. The cardiac safety risk model has at least one baseline function based on at least one baseline biomarker. The cardiac safety risk model also has at least one follow-up function based on at least one follow-up biomarker. The cardiac safety risk model further has at least one overall function based on the at least one baseline function and the at least one follow-up function.

A method of generating a cardiac safety risk model and a method of drug safety testing are also disclosed.

### BRIEF DESCRIPTION OF THE DRAWINGS

FIG. 1 schematically illustrates one embodiment of an ECG signal.

FIG. 2 illustrates one embodiment of a method for generating a personalized cardiac safety risk model.

FIG. 3 illustrates one embodiment of a method for determining a personalized cardiac safety risk factor for at least one pharmacological agent.

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

There exist numerous types of biomarkers which have been created and which will be created to quantify a measurement which is related to ventricular repolarization. Some of these biomarkers are based on an electrocardiogram. A surface electrocardiogram (ECG) may be measured by an ECG capture device which can have one or more leads which are coupled to a person's body in various locations. The electrical activity occurring within individual cells throughout the heart produces a cardiac electrical vector which can be measured at the skin's surface by the ECG capture device leads. The signal registered at the skin's surface originates from many simultaneously propagating activation fronts at different locations, each of which affects the size of the total component. One type of ECG capture device is a twelve-lead signal device, although ECG capture devices of any number of leads may be used to gather a set of ECG signals.

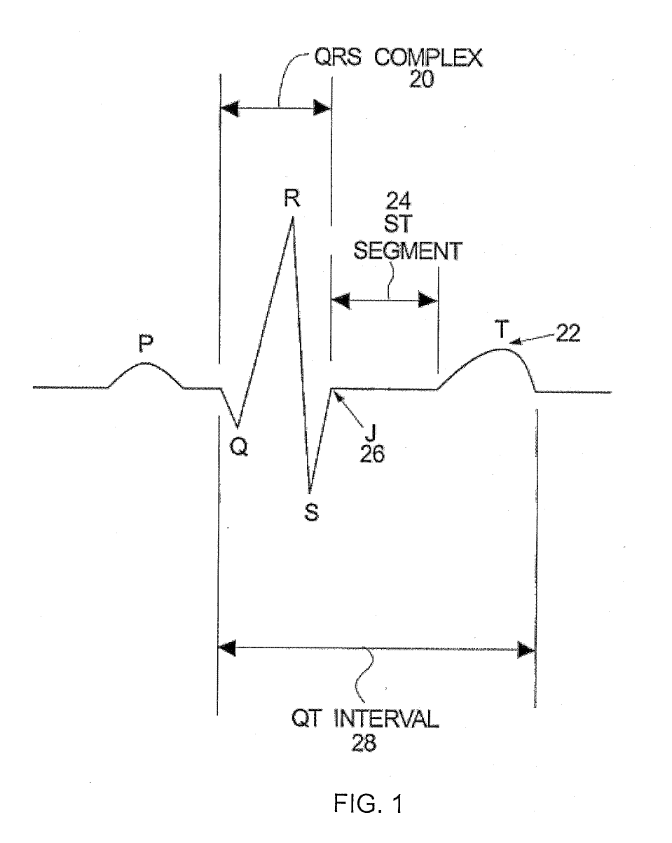

Some types of ECG-based biomarkers are simply time intervals. Various portions of an ECG waveform may be labeled by convention, and the time between various points can be measured and defined as a biomarker. For example, FIG. 1 schematically illustrates an embodiment of an ECG showing one heart beat and some of the labels which are commonly assigned to various portions of the ECG signal. The QRS complex 20 is associated with the depolarization of the heart ventricles. The QT interval 28 and the T-wave 22 are associated with repolarization of the heart ventricles. The ST segment 24 falls between the QRS complex 20 and the T-wave 22. The J point 26 is located where the QRS complex 20 joins the ST segment 24. For reference, the QT interval 28 discussed in the background and used in pre-existing FDA drug testing is illustrated. Biomarkers, such as the QT interval, may thus be arbitrarily defined. It should be apparent to those skilled in the art that other ECG interval-based biomarkers may readily be defined. Furthermore, such biomarkers may optionally be normalized or otherwise corrected, such as when the QT interval is normalized to a QTc interval by using Bazett's correction QTc=QT/RR^{0.5} or by using Fridericia's correction QTc =QT/RR^{0.33}.

Further ECG interval-based biomarkers may include, but are not limited to:

A) A beat index which indicates the beat number of a particular wave in an ECG signal; and

B) an indication of beat stability, for example a beat may be stable, or unstable based on an accelerating or decelerating heart rate.

Other types of ECG-based biomarkers are more complex in that they are based on the morphology of all or a portion of the ECG waveform. For example, some ECG morphology-based biomarkers may include a calculation of the area under a portion of the ECG curve, for example the integral of the ECG curve during the QT interval. Other examples of ECG morphology-based biomarkers may include, but are not limited to:

A) A time to reach at least one percentage of an area under the ECG signal curve, such as, for example, the method disclosed in U.S. patent application Ser. No. 10/217,883 (Publication number 2003/0050565), which is hereby incorporated by reference. In one of the embodied methods in patent application Ser. No. 10/217,883, an area under an ECG signal over a repolarization interval from a T-wave starting point to a T-wave ending point is quantified. Then, a time to reach at least one percentage of that quantified area is determined as the ECG morphology-based biomarker.

B) A repolarization duration which is based on a maximum vector which is determined from a transformed ECG signal in a plane defined by first and second eigenvectors which have been extracted from principal component analysis of at least two ECG signals, for example as disclosed in U.S. patent application Ser. No. 11/680,896, which is hereby incorporated by reference. Depending on the embodiment, early repolarization duration (ERD), late repolarization duration (LRD), and total repolarization duration (TRD) can be determined, based on a threshold percentage of the maximum vector, as the ECG morphology-based biomarker. Furthermore, the amplitude of the T-wave on the first eigenvector and the maximum vector may be used as an ECG morphology-based biomarker.

C) T-wave amplitude may be used as an ECG morphology-based biomarker.

D) T-wave peak to T-wave end (TpTe) may be used as an ECG morphology-based biomarker.

E) A right-tangent of the T-wave may be used as an ECG morphology-based biomarker.

F) A left tangent of the T-wave may be used as an ECG morphology-based biomarker.

G) An amplitude of the R-peak of the QRS complex 20 from FIG. 1 may be used as an ECG morphology-based biomarker.

H) A classification of the QRS complex 20 may be used as an ECG morphology-based biomarker. For example, the QRS complex may be classified as normal or abnormal.

While ECG-based biomarkers are useful for studying live subjects, other types of biomarkers are being developed to quantify measurements related to ventricular repolarization. For example, various patch-clamp measurement techniques may be used to measure the current response of the ion channels in a single cell or multiple cells in parallel under baseline conditions without a pharmaceutical as well as with a pharmaceutical interface with the cell(s) being tested. Patch clamp currents are an example of ion-channel based biomarkers. Another example of ion-channel-based biomarkers may be derived from fluorescent dye techniques. In these techniques, cells are treated with an indicator dye. A voltage potential is applied to the cells. The indicator dye changes hue depending on the current flowing through the cells, and this color change (and therefore ion channel current) can be recorded under various baseline and pharmaceutical conditions.

As discussed previously, various ECG-based biomarkers and ion channel biomarkers are currently used for drug safety testing and pharmacological development. Unfortunately, the characterization of drug safety risk is more complex than a single ion channel, its identification, and the response of that single channel. Furthermore, characterization of drug safety risk will vary by individual, if only because of the inherent genetic variability which is known to affect ion-channel physiology. In order to surpass the currently-accepted and suggested method of searching for the single best repolarization-related biomarker or series of biomarkers based on a generic test population, it has been posited by the applicants that a new “personalized cardiac safety risk” (PCSR) may be defined as:

$$PCSR = {f}_{Overall} [ \begin{matrix}{f}_{Baseline} ( {x}_{1 B} , {x}_{2 B} , … , {x}_{{N}_{1} B} ) \\ {f}_{Followup} ( {y}_{1 F} , {y}_{2 F} , … , {y}_{{N}_{2} F} )\end{matrix} ] \qquad ( 1 )$$

where, the PCSR (personalized cardiac safety risk) is equal to an overall function (ƒ_{Overall}) based on a combination of a plurality of functions, for example, a baseline function (ƒ_{Baseline}) and a follow-up function (ƒ_{Followup}). The baseline function is based on a plurality (one or more) of biomarkers (x_{B}) taken under baseline conditions, for example, when not in the presence of a pharmacological agent. Baseline conditions may also be determined at rest, after eating, after fasting, when hydrated, when dehydrated, while exercising, after exercising, while sleeping, at various temperatures, at a desired time before the administration of a pharmacological agent, etc. In the embodiment illustrated above, the baseline function (ƒ_{Baseline}) is based on N₁ number of biomarkers (x_{1B}, x_{2B}, . . . , x_{N1B}) taken under baseline conditions. The follow-up function (ƒ_{Followup}) is based on a plurality (one or more) of biomarkers (x_{F}) taken under follow-up conditions. Follow-up conditions may be determined during the administration of a pharmacological agent, a desired time after the administration of a pharmacological agent, and under a variety of other conditions. In the embodiment illustrated above, the follow-up function (ƒ_{Followup}) is based on N₂ number of biomarkers (y_{1F}, y_{2F}, . . . , y_{N2F}) taken under follow-up conditions.

The one or more biomarkers (x_{1B}, x_{2B}, . . . , x_{N1B}) determined under baseline conditions are preferably the same as the one or more biomarkers (y_{1F}, y_{2F}, . . . , y_{N2F}) determined under follow-up conditions. For example, in a preferred embodiment, let us assume that the one or more biomarkers being used are QTc, T-wave magnitude, and ERD at 30% of the maximum vector. In this example, these same three biomarkers could be determined under both baseline conditions and follow-up conditions. The baseline results could be plugged into a baseline function (ƒ_{Baseline}) and the follow-up results could be plugged into a follow-up function (ƒ_{Followup}). The results of these two function determinations could be plugged into an overall function (ƒ_{Overall}) to determine a personalized cardiac safety risk (PCSR).

In other embodiments, the set of one or more baseline biomarkers may be different than the set of one or more follow-up biomarkers. There may be partial overlap between the sets of biomarkers or no overlap between the sets of biomarkers.

In some embodiments, the number of baseline biomarkers N₁ will be equal to the number of follow-up biomarkers N₂, however it is not necessary. In some cases, there may be more baseline biomarkers than follow-up biomarkers and visa-versa. As a non-limiting example, some biomarkers may be more suited for providing meaningful input to the personalized cardiac safety risk function under baseline conditions because they are known to provide strong differentiation between LQT1 and LQT2 mutations when not under the influence of a pharmaceutical. However, the same biomarker may have masked or inconclusive results in the presence of pharmaceuticals, and therefore not be as useful in the follow-up function. In such a situation, in some embodiments, the biomarker in question could be left out of the follow-up determinations.

As pointed out above, the baseline biomarkers may be determined under a variety of baseline conditions, for example at rest, after eating, after fasting, when hydrated, when dehydrated, while exercising, after exercising, while sleeping, at various temperatures, at a desired time before the administration of a pharmacological agent, etc. If baseline biomarkers are taken under a plurality of baseline conditions, then in another embodiment, personalized cardiac safety risk may be defined as:

$$PCSR = {f}_{Overall} [ \begin{matrix}{f}_{Baseline 1} ( {x}_{1 {B}_{1}} , {x}_{2 {B}_{1}} , … , {x}_{{N}_{1} {B}_{1}} ) \\ {f}_{Baseline 2} ( {x}_{1 {B}_{2}} , {x}_{2 {B}_{2}} , … , {x}_{{N}_{1} {B}_{2}} ) \\ {f}_{Followup} ( {y}_{1 F} , {y}_{2 F} , … , {y}_{{N}_{2} F} )\end{matrix} ] \qquad ( 2 )$$

where, the PCSR (personalized cardiac safety risk) in the embodiment of equation (2) is equal to an overall function (ƒ_{Overall}) based on a combination of a plurality of functions, for example, a first baseline function (ƒ_{Baseline1}), a second baseline function (ƒ_{Baseline2}), and a follow-up function (ƒ_{Followup}). The baseline functions are based on a plurality (one or more) of biomarkers (x_{B}) taken under separate baseline conditions. As a non-limiting example, the first baseline function (ƒ_{Baseline1}) may be taken at rest, and the second baseline function (ƒ_{Baseline2}) may be taken while exercising. In the embodiment of equation (2), the first baseline function (ƒ_{Baseline1}) is based on N₁ number of biomarkers (x_{1B1}, x_{2B1}, . . . , x_{N1B1}) taken under the first baseline conditions. The second baseline function (ƒ_{Baseline2}) is based on N₁ number of biomarkers (x_{1B2}, x_{2B2}, . . . , x_{N1B2}) taken under the second baseline conditions. In other embodiments, a different number of baseline biomarkers may be used in the first and second baseline functions. Furthermore, although only two baseline functions have been illustrated in this example, in other embodiments, there may be any plurality of baseline functions corresponding with a variety of baseline conditions. The follow-up function (ƒ_{Followup}) is based on a plurality (one or more) of biomarkers (x_{F}) taken under follow-up conditions. Follow-up conditions may be determined during the administration of a pharmacological agent, a desired time after the administration of a pharmacological agent, and under a variety of other conditions. In the embodiment illustrated above, the follow-up function (ƒ_{Followup}) is based on N₂ number of biomarkers (y_{1F}, y_{2F}, . . . , y_{N2F}) taken under follow-up conditions.

As also pointed out above, the follow-up biomarkers may be taken under a variety of conditions, for example, during administration of a pharmacological agent or at one or more times following administration of the pharmacological agent. If follow-up biomarkers are taken under a plurality of follow-up conditions, then in another embodiment, personalized cardiac safety risk may be defined as:

$$PCSR = {f}_{Overall} [ \begin{matrix}{f}_{Baseline } ( {x}_{1 B} , {x}_{2 B} , … , {x}_{{N}_{1} B} ) \\ {f}_{Followup 1} ( {y}_{1 {B}_{1}} , {y}_{2 {B}_{1}} , … , {y}_{{N}_{2} {B}_{1}} ) \\ {f}_{Followup 2} ( {y}_{1 {B}_{2}} , {y}_{2 {B}_{2}} , … , {y}_{{N}_{2} {B}_{2}} )\end{matrix} ] \qquad ( 3 )$$

where, the PCSR (personalized cardiac safety risk) in the embodiment of equation (3) is equal to an overall function (ƒ_{Overall}) based on a combination of a plurality of functions, for example, a baseline function (ƒ_{Baseline}), a first follow-up function (ƒ_{Followup1}), and a second follow-up function (ƒ_{Followup2}). In the embodiment illustrated above, the baseline function (ƒ_{Baseline}) is based on N₁ number of biomarkers (x_{1B}, x_{2B}, . . . , x_{N1B}) taken under baseline conditions. The follow-up functions are based on a plurality (one or more) of biomarkers (y_{B}) taken under separate follow-up conditions. As a non-limiting example, the first follow-up function (ƒ_{Followup1}) may be taken at 5 minutes after administering a pharmacological agent, and the second follow-up function (ƒ_{Followup2}) may be taken 30 minutes after administering a pharmacological agent. In the embodiment of equation (3), the first follow-up function (ƒ_{Followup1}) is based on N₂ number of biomarkers (y_{1F1}, y_{2F1}, . . . , y_{N2F1}) taken under the first follow-up conditions. The second follow-up function (ƒ_{Follow-up2}) is based on N₂ number of biomarkers (y_{1F2},y_{2F2}, . . . , x_{N2F2}) taken under the second follow-up conditions. In other embodiments, a different number of follow-up biomarkers may be used in the first and second follow-up functions. Furthermore, although only two follow-up functions have been illustrated in this example, in other embodiments, there may be any plurality of follow-up functions corresponding with a variety of follow-up conditions.

In still other embodiments, the one or more baseline biomarkers may be taken under a plurality of baseline conditions while the one or more follow-up biomarkers may be taken under a plurality of follow-up conditions. Under this type of scenario, another embodiment of personalized cardiac safety risk may be defined as:

$$PCSR = {f}_{Overall} [ \begin{matrix}{f}_{Baseline 1} ( {x}_{1 {B}_{1}} , {x}_{2 {B}_{1}} , … , {x}_{{N}_{1} {B}_{1}} ) \\ {f}_{Baseline 2} ( {x}_{1 {B}_{2}} , {x}_{2 {B}_{2}} , … , {x}_{{N}_{1} {B}_{2}} ) \\ {f}_{Followup 1} ( {y}_{1 {F}_{1}} , {y}_{2 {F}_{1}} , … , {y}_{{N}_{2} {F}_{1}} ) \\ {f}_{Followup 2} ( {y}_{1 {F}_{2}} , {y}_{2 {F}_{2}} , … , {y}_{{N}_{2} {F}_{2}} )\end{matrix} ] \qquad ( 4 )$$

where all of the indicated elements of the personalized cardiac safety risk have been discussed above with regard to equations (2) and (3). It should be understood that although only two baseline functions and only two follow-up functions have been illustrated in this example, in other embodiments, there may be any plurality of baseline functions and any plurality of follow-up functions.

In a further embodiment, personalized cardiac safety risk may further and more broadly be defined as:

$$PCSR = {f}_{Overall} [ \begin{matrix}{f}_{Baseline 1} ( {x}_{1 {B}_{1}} , {x}_{2 {B}_{1}} , … , {x}_{{N}_{1} {B}_{1}} ) \\ {f}_{Baseline 2} ( {x}_{1 {B}_{2}} , {x}_{2 {B}_{2}} , … , {x}_{{N}_{1} {B}_{2}} ) \\ ⋮ \\ {f}_{Baseline α} ( {x}_{1 {B}_{α}} , {x}_{2 {B}_{α}} , … , {x}_{{N}_{1} {B}_{α}} ) \\ {f}_{Followup 1} ( {y}_{1 {F}_{1}} , {y}_{2 {F}_{1}} , … , {y}_{{N}_{2} {F}_{1}} ) \\ {f}_{Followup 2} ( {y}_{1 {F}_{2}} , {y}_{2 {F}_{2}} , … , {y}_{{N}_{2} {F}_{2}} ) \\ ⋮ \\ {f}_{Followup Ω} ( {y}_{1 {F}_{Ω}} , {y}_{2 {F}_{Ω}} , … , {y}_{{N}_{2} {F}_{Ω}} )\end{matrix} ] \qquad ( 5 )$$

where the PCSR (personalized cardiac safety risk) is equal to an overall function (ƒ_{Overall}) based on a combination of a plurality of functions, for example, at least one baseline function (ƒ_{Baseline}) and optionally up to a number of baseline functions, and at least one follow-up function (ƒ_{Followup1}) and optionally up to Ω number of follow-up functions.

A first baseline function (ƒ_{Baseline1}) is based on a plurality (one or more) of biomarkers (x_{1B1},x_{2B1}, . . . x_{N1B1}) taken under a first baseline condition B₁ where biomarker x_{1B1} is a first biomarker determined at baseline condition B₁, and biomarker x_{2B1} is an optional second biomarker determined at baseline condition B₁. Further optional biomarkers may be determined at baseline condition B₁ up-to and including optional biomarker x_{N1B1} which is an optional biomarker determined at baseline condition B₁. N₁B₁ is the total number of biomarkers taken at baseline condition B₁.

An optional second baseline function (ƒ_{Baseline2}) is based on a plurality (one or more) of biomarkers (x_{1B2},x_{2B2}, . . . x_{N1B2}) taken under a second baseline condition B₂ where biomarker x_{1B2} is a first biomarker determined at baseline condition B₂, and biomarker x_{2B2} is an optional second biomarker determined at baseline condition B₂. Further optional biomarkers may be determined at baseline condition B₂ up-to and including optional biomarker x_{N1B2} which is an optional biomarker determined at baseline condition B₂. N₁B₂ is the total number of biomarkers taken at baseline condition B₂.

Further optional baseline functions may be determined at further baseline conditions up-to and including optional baseline condition B_{α}. An α^{th} baseline function (ƒ_{Baselineα}) is based on a plurality (one or more) of biomarkers (x_{1Bα},x_{2Bα}, . . . x_{N1Bα}) taken under an α^{th} baseline condition B_{α} where biomarker x_{1Bα} is a first biomarker determined at baseline condition B_{α}, and biomarker x_{2Bα} is an optional second biomarker determined at baseline condition B_{α}. Further optional biomarkers may be determined at baseline condition B_{α} up-to and including optional biomarker x_{N1Bα} which is an optional biomarker determined at baseline condition B_{α}. N₁B_{α} is the total number of biomarkers taken at baseline condition B_{α}.

The one or more biomarkers determined for the one or more baseline functions do not all have to be the same bio markers. For example, x_{1B1}, x_{1B2}, . . . x_{1Bα} do not have to be the same biomarker. In some embodiments, however, they may be the same biomarker. Furthermore, the total number of biomarkers determined at each baseline condition B₁, B₂, . . . B_{α} do not have to be the same number, although in some embodiments, they may be the same number.

A first follow-up function (ƒ_{Followup1}) is based on a plurality (one or more) of biomarkers (y_{1F1},y_{2F1}, . . . y_{N2F1}) taken under a first follow-up condition F₁ where biomarker y_{1F1} is a first biomarker determined at follow-up condition F₁, and biomarker y_{2F1} is an optional second biomarker determined at follow-up condition F₁. Further optional biomarkers may be determined at follow-up condition F₁ up-to and including optional biomarker y_{N2F1} which is an optional biomarker determined at follow-up condition F₁. N₂F₁ is the total number of biomarkers taken at follow-up condition F₁.

An optional second follow-up function (ƒ_{Followup2}) is based on a plurality (one or more) of biomarkers (y_{1F2},y_{2F2}, . . . y_{N2F2}) taken under a second follow-up condition F₂ where biomarker y_{1F2} is a first biomarker determined at follow-up condition F₂, and biomarker y_{2F2} is an optional second biomarker determined at follow-up condition F₂. Further optional biomarkers may be determined at follow-up condition F₂ up-to and including optional biomarker y_{N2F2} which is an optional biomarker determined at follow-up condition F₂. N₂F₂ is the total number of biomarkers taken at follow-up condition F₂.

Further optional follow-up functions may be determined at further follow-up conditions up-to and including optional follow-up condition F_{Ω}. An Ω^{th} follow-up function (ƒ_{FollowupΩ}) is based on a plurality (one or more) of biomarkers (y_{1FΩ},y_{2FΩ}, . . . y_{N2FΩ}) taken under an Ω^{th} follow-up condition F_{Ω} where biomarker y_{1FΩ} is a first biomarker determined at follow-up condition F_{Ω}, and biomarker y_{2FΩ} is an optional second biomarker determined at follow-up condition F_{Ω}. Further optional biomarkers may be determined at follow-up condition F_{Ω} up-to and including optional biomarker y_{N2FΩ} which is an optional biomarker determined at follow-up condition F_{Ω}. N₂F_{Ω} is the total number of biomarkers taken at follow-up condition F_{Ω}.

The one or more biomarkers determined for the one or more follow-up functions do not have to all be the same biomarkers. For example, y_{1F1}, y_{1F2}, . . . y_{1FΩ} do not have to be the same biomarker. In some embodiments, however, they may be the same biomarker. Furthermore, the total number of biomarkers determined at each follow-up condition F₁, F₂, . . . F_{Ω} do not have to be the same number, although in some embodiments, they may be the same number.

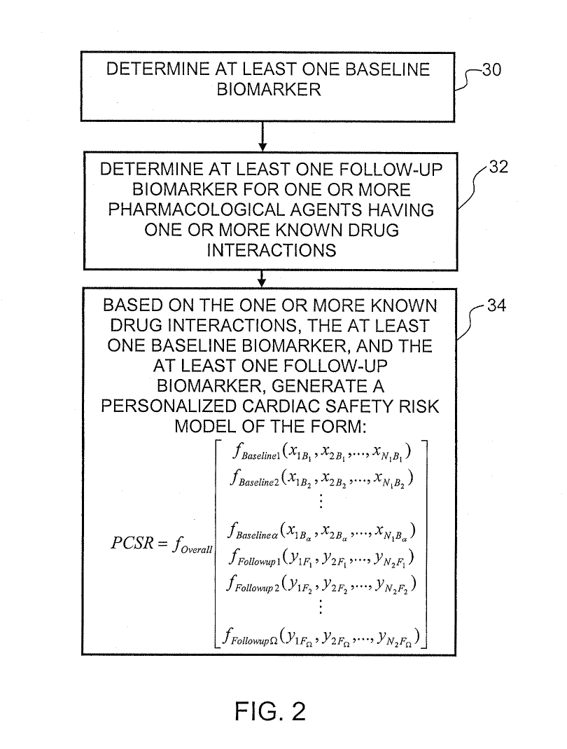

FIG. 2 illustrates one embodiment of how a personalized cardiac safety risk model may be generated. At least one baseline biomarker is determined 30. Various examples of biomarkers related cardiac repolarization have been discussed above. One or more of these types of biomarkers (ECG-based biomarkers and/or ion channel-based biomarkers) or their equivalent would be suitable for this action in the embodied method. The method should not be limited to biomarkers which exist at the time of filing, since scientists are constantly trying to develop new biomarkers. The trouble with the existing biomarker approach is that the “latest and greatest” biomarkers are always trying to outdo each other. The disclosed approach allows new biomarkers to be wrapped into a personalized cardiac safety risk model without necessarily throwing away the existing biomarkers and their associated data. At least one follow-up biomarker is determined 32 for one or more pharmaceuticals having one or more known drug interactions. As a non-limiting example, drugs which prolong QT interval or change T-wave morphology which may or may not cause torsades de pointes (TdP) can be used in-conjunction-with or prior-to the determination 32 of the at least one follow-up biomarker. Examples of drugs with known interaction data include, but are not limited to, Dofetilide, Moxifloxaxin, Sotalol, Almokalent, Cisepride, and even placebo. Larger data sets should assist the model in being more robust. Finally, based on the one or more known drug interactions, the at least one baseline biomarker, and the at least one follow-up biomarker, a personalized cardiac safety risk model is generated 34 in the form of:

$$PCSR = {f}_{Overall} [ \begin{matrix}{f}_{Baseline 1} ( {x}_{1 {B}_{1}} , {x}_{2 {B}_{1}} , … , {x}_{{N}_{1} {B}_{1}} ) \\ {f}_{Baseline 2} ( {x}_{1 {B}_{2}} , {x}_{2 {B}_{2}} , … , {x}_{{N}_{1} {B}_{2}} ) \\ ⋮ \\ {f}_{Baseline α} ( {x}_{1 {B}_{α}} , {x}_{2 {B}_{α}} , … , {x}_{{N}_{1} {B}_{α}} ) \\ {f}_{Followup 1} ( {y}_{1 {F}_{1}} , {y}_{2 {F}_{1}} , … , {y}_{{N}_{2} {F}_{1}} ) \\ {f}_{Followup 2} ( {y}_{1 {F}_{2}} , {y}_{2 {F}_{2}} , … , {y}_{{N}_{2} {F}_{2}} ) \\ ⋮ \\ {f}_{Followup Ω} ( {y}_{1 {F}_{Ω}} , {y}_{2 {F}_{Ω}} , … , {y}_{{N}_{2} {F}_{Ω}} )\end{matrix} ]$$

the features of which were discussed above with regard to equation (5). Since the drug interactions related to the one or more pharmaceuticals are known (for example a known ECG-based effect, or a known ion-channel effect) and the manifestations of these effects can be weighted as desired in terms of how much risk to cardiac safety they pose, the baseline and follow-up data can be fit into an equation of the above format using mathematical regression techniques, such as, for example the method of least squares or various Bayesian methods. Statistical techniques, such as R-Squared, analyses of the pattern of residuals, and construction of an ANOVA table may be used to check the goodness of fit of the PCSR model to the data. The biomarkers selected for use in the model may optionally be evaluated for statistical significance by various statistical techniques, such as, for example, by using an F-test and t-tests. Although both ECG-based biomarkers and/or ion channel-based biomarkers may be used in the generation of the personalized cardiac safety risk model, at least one ECG-based biomarker will preferably be used in the model. Since the model can be applied to drugs with unknown effects on an individual, the cardiac safety risk which is predicted by the model becomes personalized if it is able to be based at least in part on data which can be collected from an individual, such as with ECG-based biomarkers.

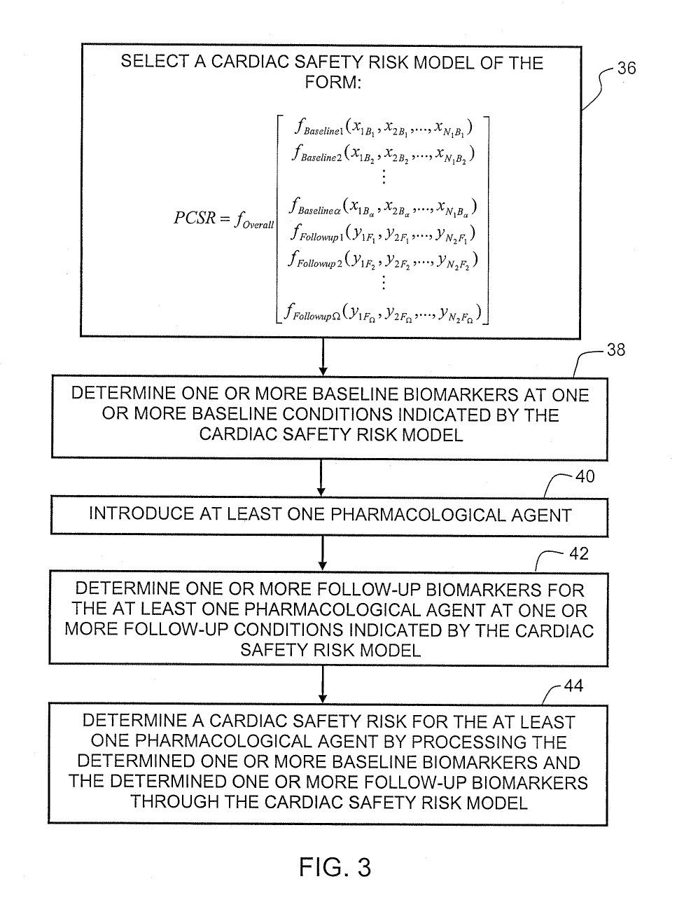

FIG. 3 illustrates one embodiment of a method of how a personalized cardiac safety risk model may be used for drug safety testing and/or pharmacological development. A personalized cardiac safety risk model of the form:

$$PCSR = {f}_{Overall} [ \begin{matrix}{f}_{Baseline 1} ( {x}_{1 {B}_{1}} , {x}_{2 {B}_{1}} , … , {x}_{{N}_{1} {B}_{1}} ) \\ {f}_{Baseline 2} ( {x}_{1 {B}_{2}} , {x}_{2 {B}_{2}} , … , {x}_{{N}_{1} {B}_{2}} ) \\ ⋮ \\ {f}_{Baseline α} ( {x}_{1 {B}_{α}} , {x}_{2 {B}_{α}} , … , {x}_{{N}_{1} {B}_{α}} ) \\ {f}_{Followup 1} ( {y}_{1 {F}_{1}} , {y}_{2 {F}_{1}} , … , {y}_{{N}_{2} {F}_{1}} ) \\ {f}_{Followup 2} ( {y}_{1 {F}_{2}} , {y}_{2 {F}_{2}} , … , {y}_{{N}_{2} {F}_{2}} ) \\ ⋮ \\ {f}_{Followup Ω} ( {y}_{1 {F}_{Ω}} , {y}_{2 {F}_{Ω}} , … , {y}_{{N}_{2} {F}_{Ω}} )\end{matrix} ]$$

is selected 36. The features of a such a model were discussed above with regard to equation (5). One or more baseline biomarkers are determined 38 at the one or more baseline conditions indicated by the personalized cardiac safety risk model. At least one pharmacological agent is introduced 40, for example by administering one or more drugs to a subject or by placing a drug solution in-line with a cell as part of a patch-clamp experiment. One or more follow-up biomarkers are determined 42 for at least one pharmacological agent at the one or more follow-up conditions indicated by the personalized cardiac safety risk model. Although the model should work very well when assessing cardiac risk for a single pharmacological agent, it may be desirable at times to test multiple pharmacological agents at the same time to assess interaction affects between the multiple agents. Furthermore, baseline and follow-up data may be gathered for an individual, a large and diverse population, or any population in-between. Finally, by crunching the determined data through the model, a personalized cardiac safety risk factor may be determined 44 for the at least one pharmacological agent.

The advantages of a method and system for determining a personalized cardiac safety risk have been discussed herein. Embodiments discussed have been described by way of example in this specification. It will be apparent to those skilled in the art that the forgoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A cardiac safety risk model for use in drug safety testing and/or pharmacological agent development, comprising:
at least one baseline function based on at least one baseline biomarker;
at least one follow-up function based on at least one follow-up biomarker; and
at least one overall function based on the at least one baseline function and the at least one follow-up function.

2. The cardiac safety risk model of claim 1, wherein the at least one baseline biomarker comprises an ECG-based biomarker.

3. The cardiac safety risk model of claim 2, wherein the ECG-based biomarker is selected from the group consisting of a QT interval, a T-wave interval, a QRS complex interval, QTc, a time to reach at least one percentage of an area under an ECG curve, ERD, LRD, TRD, T-wave amplitude, TpTe, a beat index, a right tangent of a T-wave, a left tangent of the T-wave, an R-peak amplitude, a QRS classification, a beat stability, a maximum vector, and an amplitude of the T-wave on a first eigenvector.

4. The cardiac safety risk model of claim 1, wherein the at least one baseline biomarker comprises an ion channel-based biomarker.

5. The cardiac safety risk model of claim 4, wherein the ion channel-based biomarker is selected from the group consisting of a current measured during a patch clamp experiment and a current determined during a fluorescent dye technique.

6. The cardiac safety risk model of claim 1, wherein the at least one follow-up biomarker comprises an ECG-based biomarker.

7. The cardiac safety risk model of claim 6, wherein the ECG-based biomarker is selected from the group consisting of a QT interval, a T-wave interval, a QRS complex interval, QTc, a time to reach at least one percentage of an area under an ECG curve, ERD, LRD, TRD, T-wave amplitude, TpTe, a beat index, a right tangent of a T-wave, a left tangent of the T-wave, an R-peak amplitude, a QRS classification, a beat stability, a maximum vector, and an amplitude of the T-wave on a first eigenvector.

8. The cardiac safety risk model of claim 1, wherein the at least one follow-up biomarker comprises an ion channel-based biomarker.

9. The cardiac safety risk model of claim 8, wherein the ion channel-based biomarker is selected from the group consisting of a current measured during a patch clamp experiment and a current determined during a fluorescent dye technique.

10. A method of generating a cardiac safety risk model, comprising:
determining at least one baseline biomarker;
determining at least one follow-up biomarker for one or more pharmacological agents having one or more known drug interactions; and
based on the one or more known drug interactions, the at least one baseline biomarker, and the at least one follow-up biomarker, generating a cardiac safety risk model of the form:
$$PCSR = {f}_{Overall} [ \begin{matrix}{f}_{Baseline 1} ( {x}_{1 {B}_{1}} , {x}_{2 {B}_{1}} , … , {x}_{{N}_{1} {B}_{1}} ) \\ {f}_{Baseline 2} ( {x}_{1 {B}_{2}} , {x}_{2 {B}_{2}} , … , {x}_{{N}_{1} {B}_{2}} ) \\ ⋮ \\ {f}_{Baseline α} ( {x}_{1 {B}_{α}} , {x}_{2 {B}_{α}} , … , {x}_{{N}_{1} {B}_{α}} ) \\ {f}_{Followup 1} ( {y}_{1 {F}_{1}} , {y}_{2 {F}_{1}} , … , {y}_{{N}_{2} {F}_{1}} ) \\ {f}_{Followup 2} ( {y}_{1 {F}_{2}} , {y}_{2 {F}_{2}} , … , {y}_{{N}_{2} {F}_{2}} ) \\ ⋮ \\ {f}_{Followup Ω} ( {y}_{1 {F}_{Ω}} , {y}_{2 {F}_{Ω}} , … , {y}_{{N}_{2} {F}_{Ω}} )\end{matrix} ] .$$

11. The method of claim 10, wherein determining the at least one baseline biomarker comprises determining at least one ECG-based biomarker.

12. The method of claim 11, wherein the at least one ECG-based biomarker is selected from the group consisting of a QT interval, a T-wave interval, a QRS complex interval, QTc, a time to reach at least one percentage of an area under an ECG curve, ERD, LRD, TRD, T-wave amplitude, and TpTe.

13. The method of claim 10, wherein determining the at least one follow-up biomarker comprises determining at least one ECG-based biomarker.

14. The method of claim 13, wherein the at least one ECG-based biomarker is selected from the group consisting of a QT interval, a T-wave interval, a QRS complex interval, QTc, a time to reach at least one percentage of an area under an ECG curve, ERD, LRD, TRD, T-wave amplitude, and TpTe.

15. The method of claim 10, wherein determining the at least one baseline biomarker comprises determining at least one ion channel-based biomarker.

16. The method of claim 10, wherein determining the at least one follow-up biomarker comprises determining at least one ion channel-based biomarker.

17. The method of claim 10, wherein the one or more pharmacological agents having one or more known drug interactions are selected from the group consisting of Dofetilide, Moxifloxaxin, Sotalol, Almokalent, Cisepride, and placebo.

18. The method of claim 10, wherein generating the cardiac safety risk model based on the one or more known drug interactions, the at least one baseline biomarker, and the at least one follow-up biomarker comprises performing a regression analysis.

19. The method of claim 18, wherein generating the cardiac safety risk model based on the one or more known drug interactions, the at least one baseline biomarker, and the at least one follow-up biomarker further comprises verifying a goodness of fit of the cardiac safety risk model.

20. A method of drug safety testing, comprising:
selecting a cardiac safety risk model of the form:
$$PCSR = {f}_{Overall} [ \begin{matrix}{f}_{Baseline 1} ( {x}_{1 {B}_{1}} , {x}_{2 {B}_{1}} , … , {x}_{{N}_{1} {B}_{1}} ) \\ {f}_{Baseline 2} ( {x}_{1 {B}_{2}} , {x}_{2 {B}_{2}} , … , {x}_{{N}_{1} {B}_{2}} ) \\ ⋮ \\ {f}_{Baseline α} ( {x}_{1 {B}_{α}} , {x}_{2 {B}_{α}} , … , {x}_{{N}_{1} {B}_{α}} ) \\ {f}_{Followup 1} ( {y}_{1 {F}_{1}} , {y}_{2 {F}_{1}} , … , {y}_{{N}_{2} {F}_{1}} ) \\ {f}_{Followup 2} ( {y}_{1 {F}_{2}} , {y}_{2 {F}_{2}} , … , {y}_{{N}_{2} {F}_{2}} ) \\ ⋮ \\ {f}_{Followup Ω} ( {y}_{1 {F}_{Ω}} , {y}_{2 {F}_{Ω}} , … , {y}_{{N}_{2} {F}_{Ω}} )\end{matrix} ] ;$$
determining one or more baseline biomarkers at one or more baseline conditions indicated by the cardiac safety risk model;
introducing at least one pharmacological agent;
determining one or more follow-up biomarkers for the at least one pharmacological agent at one or more follow-up conditions indicated by the cardiac safety risk model; and
determining a cardiac safety risk for the at least one pharmacological agent by processing the determined one or more baseline biomarkers and the one or more follow-up biomarkers through the cardiac safety risk model.

21. The method of claim 20, wherein determining one or more baseline biomarkers comprises determining an ECG-based biomarker or determining an ion channel-based biomarker.

22. The method of claim 20, wherein determining one or more follow-up biomarkers comprises determining an ECG-based biomarker or determining an ion channel-based biomarker.

23. The method of claim 20, wherein introducing at least one pharmacological agent comprises administering the at least one pharmacological agent to a subject.

24. The method of claim 20, wherein introducing at least one pharmacological agent comprises adding the pharmacological agent in-line with a cell as part of a patch-clamp experiment.
